(19) United States
(12) Patent Application Publication
(10) Pub. No.: US 2015/0309052 A1
(43) Pub. Date: Oct. 29, 2015

(54) ACUTE KIDNEY INJURY

(71) Applicants: Frank DIETERLE, Binningen (CH); Holger HOEFLING, Basel (CH)

(72) Inventors: Frank DIETERLE, Binningen (CH); Holger HOEFLING, Basel (CH)

(21) Appl. No.: 14/653,630

(22) PCT Filed: Dec. 18, 2013
(86) PCT No.: PCT/EP2013/077253

§ 371 Date: Jun. 18, 2015

Related U.S. Application Data
(60) Provisional application No. 61/740,303, filed on Dec. 20, 2012.

(51) Int. Cl. G01N 33/68; G01N 33/70; G06F 19/00; A61B 5/00

(52) U.S. Cl. CPC G01N 33/6893; A61B 5/7275; G01N 33/70; G06F 19/3431; G01N 2800/24; G01N 2333/54; G01N 2333/8139; G01N 2333/4703; G01N 2333/475; G01N 2333/775

(57) ABSTRACT

The present invention relates to a method of predicting the severity of acute kidney injury following cardiac surgery.

### FIELD OF THE INVENTION

The present invention relates to a method of predicting and treating acute kidney injury.

### BACKGROUND OF THE INVENTION

Acute kidney injury (AKI) is a frequent and serious complication of cardiopulmonary bypass (CPB). AKI is new or worsened renal insufficiency characterized by a relatively abrupt decrease in glomerular filtration rate (GFR), often accompanied by a reduction in urine output (Mehta et al 2007, J Vasc Surg. 46(5):1085; author reply 1085). AKI occurs most commonly following an episode of transient hypotension of any cause, but may also occur in response to nephrotoxins or radiographic contrast agents. The clinical picture of AKI may be found in 5-7% of all hospitalized patients, and may be more common in the context of complex surgery. Depending on the definition, AKI occurs in up to 3-40% of adults after cardiopulmonary bypass (CPB). Of those patients who experience AKI as a complication of cardiac surgery, the odds of death increase from four-fold for mild cases, to greater than fifteen-fold for kidney failure. Severe AKI which requires renal replacement therapy in 1-5% of cases is associated with a mortality rate of up to 70%.

The pathogenesis of CPB-associated AKI is complex and multifactorial and includes several injury pathways: diminished renal blood flow, loss of pulsatile flow, hypothermia, atheroembolism, and a generalized inflammatory response. These mechanisms of injury are likely to be active at different times with different intensities and probably act synergistically. In current clinical practice, acute kidney injury (AKI) is typically diagnosed by detecting increases in serum creatinine using various AKI definition systems such as RIFLE (risk, injury, failure, loss, end stage) or AKIN (Acute Kidney Injury Network) (Bellomo 2005 Intensive Care Med. 33(3):409-13. Epub 2006 Dec. 13., Bagshaw et al 2008 23(5):1569-74. Epub 2008 Feb. 15). However, serum creatinine is an unreliable indicator during acute changes in kidney function owing to several reasons. First, serum creatinine concentrations might not change until about 50% of kidney function has already been lost. Second, serum creatinine does not accurately reflect kidney function until a steady state has been reached, which could take several days. Finally, the serum levels of creatinine are affected by several non-renal factors such as age, gender, race, intra-vascular volume, muscle metabolism, drugs, and nutrition. All these reasons contribute to significant delays in the diagnosis of AKI and at which timepoint significant renal injury has occurred, which may be in part or in full irreversible (Bagshaw et al 2007, Curr Opin Crit Care. 13(6):638-44.). Various clinical algorithms have been proposed for the prediction of severe AKI leading to renal replacement theory (RRT), based on preoperative risk factors, but objective tests for the early diagnosis of lesser degrees of renal injury are not widely available.

There is a need to evaluate the clinical utility of biomarkers that may allow for the reliable early prediction of AKI during and after CPB, prior to the rise in serum creatinine. The ability to identify such biomarkers will help risk stratify and predict duration of acute renal failure in patients with AKI at a very early timepoint and thus result in effective preventive or therapeutic strategies.

### SUMMARY OF THE INVENTION

Currently, there has been no way to diagnose acute kidney injury (AKI) quickly (0-48 hours) in the postoperative period following cardiac surgery such as cardiopulmonary bypass surgery (CPB). The present invention not only allows for the early prediction of AKI after cardiac surgery, such as CPB, but the biomarkers of the invention can further, for the first time, be used to classify the grade of severity of AKI, enabling the administration of appropriate therapeutic interventions for those who are predicted to be at risk of developing AKI.

In one aspect, the invention includes a method of assessing the severity of acute kidney injury (AKI) injury in a subject following cardiac surgery, comprising:
measuring one or more markers from Table 1 and/or Table 2 in a biological sample obtained from the subject within 24 hours following cardiac surgery;
generating a risk score based on the measured level of one or more of the biomarkers from Table 1, wherein if the risk score exceeds a predefined cutoff, the subject is determined to be at risk of developing RIFLE I/F; and
optionally, if the subject is not determined to be at risk of developing RIFLE I/F, further generating a risk score based on the measured level of one or more of the biomarkers selected from Table 2, wherein if the risk score exceeds a predefined cutoff, the subject is determined to be at risk of developing RIFLE R, or if the risk score is below the predefined cutoff, the subject is determined not to be at risk of developing AKI.

In one example, two, three, four or more biomarkers from Table 1 are measured to determine if the subject is at risk of developing RIFLE I/F. In another example, two or three biomarkers from Table 2 are measured to determine if the subject is at risk of developing RIFLE R. In another example, two or more biomarkers from Table 1 and Table 2 are measured to determine if the subject is at risk of developing RIFLE I/F or RIFLE R or no AKI.

Examples of single markers and combinations that can be used to determine if the subject is at risk of developing RIFLE I/F are shown in Table 14. Examples of combinations that can be used to determine if the subject is at risk of developing RIFLE R are shown in Table 15. Examples of other combinations are shown in Table 3.

In another aspect, the invention includes a method of assessing the severity of acute kidney injury (AKI) injury in a subject following cardiac surgery, comprising:
measuring TFF3 in a biological sample obtained from the subject within 24 hours following cardiac surgery;
generating a risk score based on the measured level of the biomarker wherein the risk score when compared to a predefined cutoff is indicative if the subject is at risk of developing RIFLE I/F.

In another aspect, the invention includes a method of assessing the severity of acute kidney injury (AKI) injury in a subject following cardiac surgery, comprising:
measuring A1-microglobulin in a biological sample obtained from the subject within 24 hours following cardiac surgery;
generating a risk score based on the measured level of the biomarker wherein the risk score when compared to a predefined cutoff is indicative if the subject is at risk of developing RIFLE I/F.

In another aspect, the invention includes a method of assessing the severity of acute kidney injury (AKI) injury in a subject following cardiac surgery, comprising:
measuring at least one of the following biomarkers selected from the group consisting of IL-18, Cystatin C, NGAL, TFF3, Clusterin, B2-Microglobulin and A1-Microglobulin in a biological sample obtained from the subject within 24 hours following cardiac surgery;
generating a risk score based on the measured level of one or more of the biomarkers wherein the risk score when compared to a predefined cutoff is indicative if the subject is at risk of developing RIFLE I/F, RIFLE R or is not at risk of AKI.

In yet another aspect, the invention includes a method of assessing the severity of acute kidney injury (AKI) injury in a subject following cardiac surgery, comprising:
measuring at least one of the following biomarkers selected from the group consisting of IL-18, Cystatin C, NGAL, TFF3, Clusterin, and A1-Microglobulin in a biological sample obtained from the subject within 24 hours following cardiac surgery;
generating a risk score based on the measured level of one or more of the biomarkers wherein the risk score is indicative if the subject is at risk of developing RIFLE I/F.

In still yet another aspect, the invention includes a method of assessing the severity of acute kidney injury (AKI) injury in a subject following cardiac surgery, comprising:
measuring at least one of the following biomarkers selected from the group consisting of TFF3, B2-microglobulin and A1-microglobulin in a biological sample obtained from the subject within 24 hours following cardiac surgery;
generating a risk score based on the measured level of one or more of the biomarkers, wherein the risk score is indicative if the subject is at risk of developing RIFLE R or is not at risk of developing AKI.

In another aspect, the invention includes a method of diagnosing or predicting development of acute kidney injury (AKI) in a subject following cardiac surgery, comprising measuring at least four of the following biomarkers selected from IL-18, Cystatin C, NGAL, TFF3, Clusterin, B2-microglobulin and A1-Microglobulin in a biological sample obtained from the subject within 24 hours following cardiac surgery; wherein the levels are indicative of AKI or are predictive of the development of AKI.

In another aspect, the invention includes a method of diagnosing or predicting development of acute kidney injury (AKI) in a subject following cardiac surgery, comprising measuring any of the following:
TFF3 and at least one of the following biomarkers selected from IL18, Cystatin C, NGAL, Clusterin, B2-microglobulin and A1-Microglobulin in a biological sample obtained from the subject within 24 hours following cardiac surgery, wherein the levels are indicative of AKI or are predictive of the development of AKI;
A1-microglobulin and at least one of the following biomarkers selected from IL18, Cystatin C, NGAL, Clusterin, B2-microglobulin, and TFF-3 in a biological sample obtained from the subject within 24 hours following cardiac surgery, wherein the levels are indicative of AKI or are predictive of the development of AKI; or
clusterin and at least one of the following biomarkers selected from IL18, Cystatin C, NGAL, A1-microglobulin, B2-microglobulin and TFF-3 in a biological sample obtained from the subject within 24 hours following cardiac surgery, wherein the levels are indicative of AKI or are predictive of the development of AKI.

In the methods described above, urinary creatinine (uCr) can also be measured in the subject following cardiac surgery such as CPB surgery and a ratio of each of the markers with uCr as a predictor of the development of acute kidney injury (AKI) in the subject. In one example, a weighted linear combination of at least one biomarker/uCr is used with Receiver-Operating Characteristic (ROC) area under the curve analysis is used to predict development and severity of AKI in the subject.

In yet another aspect, the invention includes a diagnostic kit for quantitative measurement of one or more biomarkers shown in Table 1 and Table 2 in a sample of a patient which has been taken within 24 hours following cardiac surgery, wherein the level of the biomarkers is indicative as to whether the subject will develop AKI and the severity of AKI.

The biomarkers of the invention can be measured using any device or method known in the art. In one example, a point of care device for diagnosing or predicting development of acute kidney injury (AKI) in a subject following cardiac surgery is used. In one example, the device will be used to measure at least one marker from Table 1 and one marker from Table 2 in a biological sample obtained from the subject within 24 hours following cardiac surgery; wherein the levels are indicative of AKI and the severity of AKI. Examples of cardiac surgery include CPB.

### BRIEF DESCRIPTION OF THE DRAWINGS

FIG. 1 depicts a boxplot of IL-18 values after urinary creatinine normalization for different time points before and after surgery.

FIG. 2 depicts a boxplot of NGAL values after urinary creatinine normalization for different time points before and after surgery.

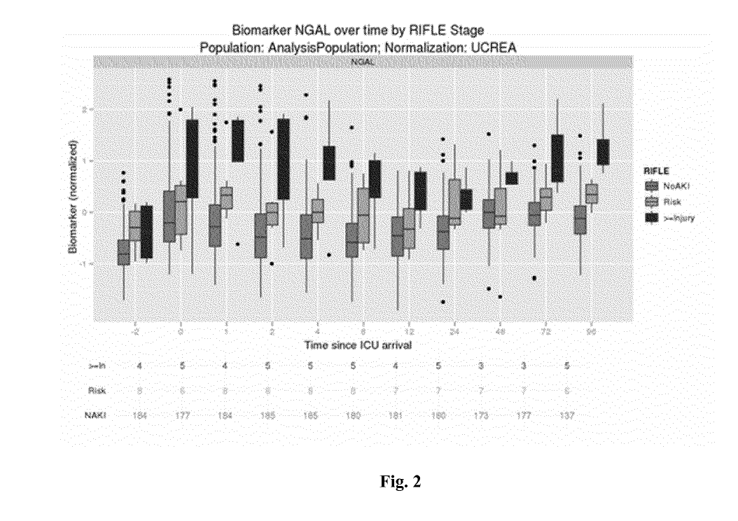

FIG. 3 depicts a boxplot of TFF3 values after urinary creatinine normalization for different time points before and after surgery.

### DETAILED DESCRIPTION OF THE INVENTION

There is an increasing body of evidence that suggests a patient's genetic and protenomic profile can be used for diagnosis of diseases or can be determinative of a patient's responsiveness to a therapeutic treatment. Given the numerous therapies available to treat various diseases, a determination of the genetic and protein factors that can be used to predict or influence, for example, patients response to a particular surgery or drug. The determination of these factors could be used to provide better treatment and early intervention.

A serious complication of cardiopulmonary bypass surgery (CPB) is acute kidney injury (AKI) which refers to a rapid loss of kidney function. AKI after CPB has an incidence rate of 3-40% and is a serious complication due to its late diagnosis (typically 1-5 days after the event) that can often lead to increased mortality and risk of chronic kidney disease. To establish a uniform definition for acute kidney injury, the Acute Dialysis Quality Initiative formulated the Risk, Injury, Failure, Loss, and End-stage Kidney (RIFLE) classification.

RIFLE defines three grades of increasing severity of acute kidney injury—risk (class R), injury (class I) and failure (class F). The RIFLE classification provides three grades of severity for acute kidney injury based on changes in either serum creatinine or urine output from the baseline condition. For example, the following serum creatinine (SCr) levels compared to baseline can be used to stage patients:

Risk: SCr increased 1.5 times relative to baseline

Injury: SCr increased 2 times relative to baseline

Failure: SCr increased 3 times relative to baseline

Diagnosis of AKI only based on serum creatinine (SCr) has limitations including variability of SCr measurement can be influenced by patient hydration status or fluid management. Also, SCr is not very sensitive and often occurs only 1-5 days after injury has occurred. Some patients who have a good renal baseline function the kidney injury can occur without an increase of SCr due to “renal reserve”. Urine output, which is another element of the RIFLE for AKI is similar to SCr late and not sensitive, in particular for AKI after CPB. Currently practiced methods thus for diagnosing and grading AKI are inadequate. The present invention allows for the early prediction of AKI after cardiac surgery such as CPB surgery and offers the potential to maximize therapeutic benefit to CPB patients who will develop AKI.

The methods described herein are based, in part, upon the identification of a single or a plurality of protein biomarkers in the urine which can be used to predict early (e.g., within 24 hours) whether a patient following cardiac surgery will develop AKI and in particular to predict the severity of AKI. According to the present invention, while trying to conform with the present recognized system for grading AKI using RIFLE, the present invention can be used to classify patients into three grades. Specifically, the biomarkers of the invention can predict whether an individual would likely following surgery develop RIFLE risk class I or F (herein referred to as RIFLE I/F). If the individual is determined not to have RIFLE I/F, the individual can further be assessed for the likelihood of that individual developing RIFLE R. If the individual is assessed not to fall into the RIFLE R category, then the individual is assessed to be an individual that is unlikely to develop AKI.

Thus, the present methods provide a means of predicting whether an individual is likely to develop RIFLE I/F, RIFLE R or no AKI.

The methods of the present invention are not just applicable to cardiac surgery such as CPB or CABG but to any surgery (physical trauma) or event where AKI might result and a determination of the level of severity of AKI would be beneficial. Surgeries contemplated can include heart and transplant surgeries and others.

Biomarker

The present invention is based on the finding that particular protein biomarkers can be used to indicate and grade AKI within 48 hours (such as 0.5, 1, 2, 3, 4, 5, 6, 7, 8, 9, 10, 11, 12, 20, 24, 28, 30, 34, 38, 40, 42, 44, 46, or 48 hours) following cardiac surgery such as CPB. In particular it was found that kidney biomarkers could be divided into two groups so that three groups of severity of AKI could be predicted as explained above. The first group of biomarkers are indicative of severe AKI (equivalent to “Injury” and “failure” as interpreted by the RIFLE model; RIFLE I/F) and are shown in Table 1 and the second group of markers are indicative of more moderate AKI (equivalent to “Risk” as interpreted by the RIFLE, RIFLE R) and shown in Table 2.

In one example, a single biomarker such as TFF3 or A1-Microglobulin can be used to determine if an individual is at risk of developing RIFLE I/F by generating a risk score and comparing the risk score to a predefined cutoff.

In another example, a single biomarker such as TFF3 or A1-Microglobulin can be used to determine first if an individual is at risk of developing RIFLE I/F by generating a risk score and comparing the risk score to a predefined cutoff, and if the individual is determined not to have RIFLE I/F, the single markers can optionally also be used to determine if that individual is at risk of developing RIFLE R, by generating a risk score and comparing the risk score to a predefined cutoff. If the individual is determined not to have RIFLE I/F or RIFLE R, then that individual is assessed as not having any risk of developing AKI.

In another example, it was found that a combination of an RIFLE I/F biomarker (Table 1) and/or a RIFLE R biomarker (Table 2) can be used to predict and grade the severity of AKI within 48 hours, e.g., 12, 8, 4 hours or less following CPB.

TABLE 1

| | Changes of | Swiss Prot |
|---|---|---|
| RIFLE I/F biomarker | levels upon AKI | Accession # |
| IL-18 | Upregulated | Q14116 |
| Cystatin C | Upregulated | P01034 |
| NGAL | Upregulated | P80188 |
| TFF3 (Trefoil factor 3) | Upregulated | Q07654 |
| Clusterin | Upregulated | P10909 |
| A1-Mic (α-1-Microglobulin) | Upregulated | P02760 |

TABLE 2

| | Changes of | Swiss Prot |
|---|---|---|
| RIFLE R Biomarker | levels upon AKI | Accession # |
| TFF3 (Trefoil factor 3) | Upregulated | Q07654 |
| β-2 M (β-2 Microglobulin) | Upregulated | P61769 |
| A1-Mic (α-1-Microglobulin) | Upregulated | P02760 |

In another example, the biomarker(s) of the invention includes at least one biomarker protein listed in Table 1 and at least one biomarker protein listed in Table 2. Any combination of biomarkers can be selected. Examples of combinations are shown in Table 3 below.

TABLE 3

| Combinations | Examples |
|---|---|
| 1 | IL-18 and A1-Mic |
| 2 | IL-18, Cys C, and A1-Mic |
| 3 | IL-18, Cys C, NGAL, and A1-Mic |
| 4 | Cys C, NGAL and A1-Mic |
| 5 | Cys C, NGAL, TFF3 and A1-Mic |
| 6 | NGAL, TFF3 and A1-Mic |
| 7 | NGAL, TFF3, Clusterin, and A1-Mic |
| 8 | TFF3 and A1-Mic |
| 9 | TFF3, Clusterin and A1-Mic |
| 10 | Clusterin and A1-Mic |
| 11 | IL18, Cys C, NGAL, TFF3, Clusterin, A1-Mic and TFF3 |
| 12 | Clusterin and A1-Mic; |
| 13 | IL18, Cys C, NGAL, and TFF3 |
| 14 | Clusterin, A1-Mic and TFF3 |
| 15 | Cys C, NGAL, TFF3, Clusterin, and A1-Mic |
| 16 | TFF3, Clusterin, A1-Mic and B2-Mic |

Detection of Biomarker Proteins

The biomarker proteins disclosed in Table 1 and Table 2 are measured to make a determination of whether a subject following cardiac surgery such as CPB has an increased likelihood of developing a particular grade of AKI. Typically the methods of the invention are used to detect the biomarker protein of interest in a biological fluid sample of interest such as urine, blood, serum, or plasma. In one example, the RIFLE I/F markers identified in Table 1 or the RIFLE R markers identified in Table 2 are measured from a serum or plasma sample in a patient following cardiac surgery and the serum levels are used to predict development and severity of AKI as determined by RIFLE criteria discussed above. In another example, the RIFLE I/F biomarkers identified in Table 1 or the RIFLE R biomarkers identified in Table 2 are measured from a urine sample in a patient following cardiac surgery and the urine levels are used to predict development and severity of AKI. Optionally serum creatinine (sCr) and/or urinary creatinine (uCr) in the patient following the event can also be measured and used for normalisation.

The biological samples used in the practice of the inventive methods may be fresh or frozen samples collected from a subject, or archival samples with known diagnosis, treatment and/or outcome history. In certain embodiments, the inventive methods are performed on the urine sample itself without or with limited processing of the sample.

In some examples, the biomarker proteins of interest can be measured pre-operatively, e.g., between 0-24 hours preoperatively and/or within 48 hours, e.g., just after surgery, time 0, at or any time thereafter including any time between 0-0.5, between about 0-1, between about 0-2, between about 0-3, between about 0-4, between about 0-5, between about 0-6, between about 0-7, between about 0-8, between about 0-9, between about 0-10; or between about 0.5-4 hours; or between about 0.5-8 hours; or between about 0.5-12 hours; or between about 0.5-24 hours; or between about 0.5-48 hours; or about 0.5 hours; or about 1 hour; or about 2 hours; or about 3 hours; or about 4 hours; or about 5 hours; or about 6 hours; or about 7 hours; or about 8 hours; or about 9 hours; or about 10 hours; or about 11 hours; or about 12 hours; or about 24 hours following surgery (e.g., CPB). In another example, the biomarker proteins of interest can be measured following admittance into the ICU. In this description, “about” is employed in quantitative terms to denote a range of plus-or-minus 10 percent. Moreover, where “about” is used in conjunction with a quantitative term, it is understood that, in addition to the value plus or minus 10 percent, the exact value of the quantitative term also is contemplated and described. For instance, the term “about 3 percent ” expressly contemplates, describes, and includes exactly 3 percent.

The biomarker levels described herein can be directly calculated or can be calculated and/or expressed as a ratio with a normalization biomarker such as creatinine (or any other appropriate markers). For example, TFF3 levels may be calculated and/or expressed as a ratio of creatinine levels in the same sample type (for example the levels may be expressed as ng TFF3 per milliliter of urine divided by urinary creatinine expressed as mg/ml urine).

The method of the invention can also include measuring the urine biomarker of Table 1 or Table 2 and using the kinetics of the change in the presence of the biomarker following the event to predict development and severity of AKI in the patient. In fact, biomarkers were specifically chosen based on their dynamic range, i.e. biomarkers whose levels are strongly modulated upon injury compared to baseline levels before injury or compared to levels in non-AKI subjects (normal ranges) are preferred. See also example 7.

In one embodiment, where the kinetics of change are being measured, a positive percent change is associated with RIFLE R AKI and a more positive percent change is predictive of RIFLE I/F.

A urinary biomarker protein level can be measured using any assay known to those of ordinary skilled in the art, including, but not limited to, immunoprecipitation assays, mass spectrometry, Western Blotting, and via dipsticks using conventional technology. In one embodiment, the levels of biomarker proteins in urine are detected by an immunoassay Immunoassays include but are not limited to enzyme immunoassay (EIA), also called enzyme-linked immunosorbant assay (ELISA), radioimmunoassay (RIA), diffusion immunoassay (DIA), fluoroimmunoas say (FIA), chemiluminescent immunoassay (CLIA), counting immunoassay (CIA), lateral flow tests or immunoassay (LFIA), also known as lateral flow immunochromatographic assays, and magnetic immunoassay (MIA).

For purposes of comparison, the levels of a biomarker protein in a urine sample from the patient can be measured against measured urinary Cr levels, which is used as a normalization value.

The levels of a biomarker of Table 1, which is used to predict whether an individual is likely to develop RIFLE I/F risk, or the levels of biomarker of Table 2, which is used to measure whether an individual is likely to develop RIFLE R, in a sample such as urine, can be determined using any protein-binding agent. In some embodiments, a protein-binding agent is a ligand that specifically binds to a biomarker protein, and can be for example, a synthetic peptide, chemical, small molecule, or antibody or antibody fragment or variants thereof. In some embodiments, a protein-binding agent is a ligand or antibody or antibody fragment, and in some embodiments, a protein-binding agent is preferably detectably labeled.

In one embodiment of the invention, immunoassays using antibodies are used to measure the levels of biomarker proteins of Table 1 and/or Table 2 in urine. As used herein, the term “antibody” includes polyclonal, monoclonal, or other purified preparations of antibodies and recombinant antibodies includes humanized antibodies, bispecific antibodies, and chimeric molecules having at least one antigen binding determinant derived from an antibody molecule. Antibody as used is intended to include whole antibodies, e.g., of any isotype (IgG, IgA, IgM, IgE, etc), and includes fragments thereof which are also specifically reactive with the biomarker proteins to be measured. Non limiting examples of fragments of antibodies include proteolytic and/or recombinant fragments such as Fab, F(ab′)2, Fab′, Fv, dAbs and single chain antibodies (scFv) containing a VL and VH domain joined by a peptide linker. The scFv's can be covalently or non-covalently linked to form antibodies having two or more binding sites.

The biomarker proteins useful in the methods of the invention are known in the art.

TABLE 4

| Protein | Entrez | | Accession | |
|---|---|---|---|---|
| symbol | ID | Description | (UniProt) | Symbol Alias |
| A1-Mic | 259 | alpha-1- | P02760 | AMBP; A1M; EDC1; |
| | | microglobulin | | HCP; HI30; IATIL; ITI; |
| | | | | ITIL; ITILC; UTI |
| B2M | 567 | beta-2- | P61769 | B2M |
| | | microglobulin | | |
| CLU | 1191 | clusterin | P10909 | CLU; APO-J; APOJ; CLI; |
| | | | | KUB1; NA1/NA2; SGP-2; |
| | | | | SGP2; SP-40; TRPM-2; |
| | | | | TRPM2 |
| CYS C | 1471 | cystatin C | P01034 | CST3; ARMD11 |
| IL18 | 3606 | interleukin 18 | Q14116 | IL18; IGIF; IL-18; IL-1g; |
| | | | | IL1F4 |
| NGAL | 3934 | lipocalin 2 | P80188 | LCN2; 24p3; MSFI; |
| | | | | NGAL |
| TFF3 | 7033 | trefoil factor 3 | Q07654 | TFF3; ITF; P1B; TFI |

Antibodies to the biomarker proteins can be generated using methods known to those skilled in the art. Alternatively, commercially available antibodies can be used. In one embodiment, commercial kits for assaying the biomarkers of interest are available, e.g., RBM.

In one embodiment, the antibody is detectably labeled.

As used herein “detectably labeled”, includes antibodies that are labeled by a measurable means and include, but are not limited to, antibodies that are enzymatically, radioactively, fluorescently, and chemiluminescently labeled. Antibodies can also be labeled with a detectable tag, such as c-Myc, HA, VSV-G, HSV, FLAG, V5, HIS, or biotin.

In one embodiment, the antibody is detectably labeled by linking the antibody to an enzyme. The enzyme, in turn, when exposed to it's substrate, will react with the substrate in such a manner as to produce a chemical moiety which can be detected, for example, by spectrophotometric, fluorometric, or by visual means. Enzymes which can be used to detectably label the antibodies of the present invention include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase.

It is also possible to label an antibody with a fluorescent compound. When the fluorescently labeled antibody is exposed to light of the proper wave length, its presence can then be detected due to fluorescence. Among the most commonly used fluorescent labeling compounds are CYE dyes, fluorescein isothiocyanate, rhodamine, phycoerytherin, phycocyanin, allophycocyanin, o-phthaldehyde and fluorescamine An antibody can also be detectably labeled using fluorescence emitting metals such as labels of the lanthanide series. These metals can be attached to the antibody using such metal chelating groups as diethylenetriaminepentaacetic acid (DTPA) or ethylenediaminetetraacetic acid (EDTA).

An antibody also can be detectably labeled by coupling it to a chemiluminescent compound. The presence of the chemiluminescent-antibody is then determined by detecting the presence of luminescence that arises during the course of a chemical reaction. Examples of particularly useful chemiluminescent labeling compounds are luminol, luciferin, isoluminol, theromatic acridinium ester, imidazole, acridinium salt and oxalate ester.

In one example, the assay used to determine the level of RIFLE I/F and RIFLE R are immunoassays such as a competitive immunoassay. In another embodiment, the immunoassay is a noncompetitive immunoassay.

In another embodiment, the levels of biomarker proteins in urine are detected by ELISA assay. There are different forms of ELISA which are well known to those skilled in the art, e.g. standard ELISA, competitive ELISA, and sandwich ELISA. The standard techniques for ELISA are described in “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, M. 1984, J. Clin. Chem. Clin. Biochem., 22:895-904.

For the ELISA method described herein a known amount of anti-biomarker antibody is affixed to a solid surface, and then the urine sample containing the biomarker of interest is washed over the surface so that the antigen biomarker can bind to the immobilized antibodies (a first antibody). The surface is washed to remove any unbound biomarker and also any non-biomarker proteins present in the urine sample. A detection antibody (a second antibody) is applied to the surface. The detection antibody is specific to the biomarker in the subject. Performing an ELISA involves a known amount of anti-biomarker antibody being immobilized on a solid support (usually a polystyrene micro titer plate) either non-specifically (via adsorption to the surface) or specifically (via capture by another antibody specific to the anti-biomarker antibody, in a “sandwich” ELISA). After the biomarker protein from the sample is immobilized, the detection antibody is added, forming a complex with the antigen.

In one embodiment, the levels of at least one biomarker from Table 1 and at least one biomarker from Table 2 are selected and measured using at least two antibodies specific to each biomarker protein to be measured. In another embodiment, the levels of three biomarker proteins (at least one is chosen from Table 1 and one from Table 2) at one defining a first biomarker protein, a second biomarker protein, and a third biomarker protein, are measured using at least three antibodies specific to each biomarker protein to be measured, wherein each antibody specifically reacts with the first biomarker protein, the second biomarker protein, or the third biomarker protein to be measured. In one embodiment, the levels of four biomarker proteins (at least one is chosen from Table 1 and one from Table 2) defining a first, a second, a third and a fourth biomarker protein, are measured using at least four antibodies specific to each biomarker protein to be measured.

In another embodiment, the levels of biomarkers in Table 1 and/or Table 2 in a sample are detected by an on-the-spot assay also known as point-of-care test (POC). POC is defined as diagnostic testing at or near the site of patient care such as in this case the POC could be in the ICU. As evidenced by the examples provided, the present invention can provide an accurate read as to the patient's status with respect to developing and grading RIFLE I/F, or RIFLE R, or no AKI, within the first 1-24 hours following cardiac surgery. POC brings the test conveniently and immediately to the patient. This increases the likelihood that the patient will receive the results in a timely manner. POC is accomplished through the use of transportable, portable, and handheld instruments (e.g., blood glucose meter, nerve conduction study device) and test kits (e.g., CRP, HBA1C, Homocystein, HIV salivary assay, etc.). POC tests are well known in the art, especially immunoassays. For example, the LFIA test strip or dip sticks can easily be integrated into a POC diagnostic kit. One skilled in the art would be able to modify immunoassays for POC using different format, e.g. ELISA in a microfluidic device format or a test strip format.

In one embodiment, the levels of biomarker proteins in urine are detected by a lateral flow immunoassay test (LFIA), also known as the immunochromatographic assay, or strip test. LFIAs are a simple device that can detect the proteins in Table 1 and/or Table 2 to detect the presence (or absence) of a target biomarker antigen in a fluid sample. There are currently many LFIA tests are used for medical diagnostics either for home testing, point of care testing, or laboratory use. LFIA tests are a form of immunoassay in which the test sample flows along a solid substrate via capillary action. After the sample is applied to the test it encounters a colored reagent which mixes with the sample and transits the substrate encountering lines or zones which have been pretreated with an antibody or antigen.

In another embodiment, the levels of biomarker proteins in urine are detected by a diffusion immunoassay (DIA). In this assay, the transport of molecules perpendicular to flow in a microchannel, e.g. in a microfluidic chip, is affected by binding between antigens and antibodies. Microfluidic diffusion immunoassays for the detection of analytes or biomarkers in fluid samples have been described in the art, for example, in U.S. Pat. Nos. 6,541,213; 6,949,377; 7,271,007; U.S. Patent Application No. 20090194707; 20090181411; in Hatch et al., 2001, Nature Biotechnology 19(5): 461-465; K.

In another example, the POC test device is based on a piezo (or pyro) film which is disclosed in US20060263894, which is incorporated herein by reference. In one embodiment using this POC test, the piezofilm is coated with antibody directed against one or more biomarker(s) disclosed in Table 1 and/or Table 2 of the present invention. In one example, the POC device is a cartridge having a capillary tube leading to a chamber in which the piezofilm sits. The inside surface of the capillary tube is coated with a dried-down layer of a second antibody directed against one or more biomarker(s) disclosed in Table 1 and/or Table 2 of the present invention (this time linked to carbon particles) also specifically able to bind the biomarker(s) disclosed in Table 1 and/or Table 2 but at a different molecular site from the antibodies bound to the piezofilm. The bodily fluid sample moves along the capillary tube, dissolving the carbon-antibody-conjugate, to the piezofilm test area within the cartridge. Once the sample, mixed with the carbon conjugates, reaches the piezofilm, the one or more protein biomarker(s) disclosed in Table 1 and/or Table 2 of the present invention, if present in the sample being tested, binds to both antibodies at the same time. The reaction results in a “sandwich” in which the one or more biomarker(s) disclosed in Table 1 and/or Table 2 of the present invention is compressed between the two sets of antibodies. The sandwich reaction causes the carbon particles to become linked to the piezofilm. During the reaction, a desktop reader illuminates the sample every few milliseconds using a flashing light-emitting diode (LED). Carbon particles linked to the film absorb the light and convert it to heat which deforms the film to generate a charge. As more carbon particles become linked to the film, each pulse of light results in greater heat transfer and so greater charge. The rate of change of charge is proportional to the concentration of the one or more biomarker(s) disclosed in Table 1 and/or Table 2 of the present invention in the sample. The measurement of charge over time across the piezofilm measures the protein biomarkers concentration in the sample.

In another embodiment using the system described above a competitive assay format can be employed. In this example, the antibody against one or more of the biomarkers listed in Table 1 and/or Table 2 is coated onto the piezo film and the inside of the capillary tube is coated with a dried-down layer of the biomarker protein derivative conjugated to a carbon label. Once the bodily fluid sample moves along the capillary tube it dissolves the carbon-protein-conjugate. Once the sample, mixed with the carbon conjugates, reaches the piezofilm, the biomarker protein in the sample competes with the protein conjugate for the coated biomarker antibody and the concentration of the protein biomarker can be determined by measuring change over time across the piezofilm. Alternatively, a biomarker derivative to which the sample protein and an antibody can bind of one or more of the biomarkers shown in Table 1 and/or Table 2 is bound to the piezofilm. In this example, the inside surface of the capillary tube is coated with a dried-down layer of the biomarker antibody labeled with carbon. Once the sample dissolves the antibody-carbon conjugate, the biomarker protein in the sample compete with the biomarker derivative for binding to the antibody. Concentration of the protein biomarker can be determined by measuring change over time across the piezofilm. The competitor used in these assays can be any molecule, peptide or derivate thereof which can compete with the biomarker protein for the biomarker antibody binding site. The biomarker derivative can be conjugated to any known label, including e.g., biotinylated or carbon.

Kits

Embodiments of the invention further provide for diagnostic kits and products of manufacture comprising the diagnostic kits. The kits can comprise a means for predicting AKI in a human.

In one embodiment, the kit comprises an indicator responsive to the level of biomarker protein in a sample of urine, wherein the biomarker protein is selected from at least one biomarker from Table 1 and at least one biomarker from Table 2. See Table 3 for examples. The kits can further include cups or tubes, or any other collection device for sample collection of urine. In another embodiment, the kit can optionally further comprise at least one diagram and/or instructions describing the interpretation of test results.

Data Analysis

In the methods of the invention, the level of each biomarker measured will typically be converted into a value after normalization with uCR or the average of one or several control proteins or endogenous metabolites or specific urine gravity. The values generated will then be provided to a AKI software algorithm and used to generate a score which is then compared against a predefined cut-off to select subjects that are likely to develop AKI and predict the severity of AKI.

In one example, a weighted linear combination of at least one biomarker of Table 1/uCr and one biomarker of Table 2/uCr is used with Receiver-operating characteristic (ROC) area under the curve analysis to predict development of AKI in the subject.

To facilitate the sample analysis operation, the data obtained by the reader from the device may be analyzed using a digital computer. Typically, the computer will be appropriately programmed for receipt and storage of the data from the device, as well as for analysis and reporting of the data gathered, for example, subtraction of the background, verifying that controls have performed properly, normalizing the signals, interpreting fluorescence data to determine the amount of hybridized target, normalization of background, and the like.

In one example, in the methods of the invention, urine samples from a patient undergoing cardiac surgery such as CPB surgery will be collected after surgery and optionally also collected before surgery as a baseline. The urine samples will be measured for any of the biomarkers set out in Table 1 and/or 2 for the post-surgery sample and optionally, for the baseline samples. Urinary creatinine may also be measured to normalize the levels of the biomarkers of the invention. The data can be analyzed by any method in the art including those methods set out below:

Method 1: Only Preprocessing

Step 1: Measure one or more of the biomarkers in Table 1 and Table 2 pre and post surgery.

Step 2: Each of the processed measurements of biomarkers in Table 1 are compared to marker-specific cutoffs. The number of markers that exceed the marker-specific cutoff will be determined. If a pre-specified number of markers exceed the cutoff, the patient will be classified as belonging to the RIFLE I/F category. It may be that it is required that all markers exceed the cutoff, or all but one marker, or all but two markers etc., or only a single marker exceeds the cutoff If the patient is classified as RIFLE I/F, the evaluation stops here, otherwise, the evaluation may proceed at the next step.

Step 3: Take a weighted average of all processed marker measurements of biomarkers in Table 2 and compare the result to a pre-specified cutoff. The weights used may be the same for all biomarkers, however they may also be specific for each marker. If the weighted average is above the cutoff, classify the result as RIFLE R. If the patient is not classified as RIFLE R, go to the next step.

Step 4: Classify the patient as “No AKI”.

Method 2: Pre-Processing and Urinary Creatinine Normalization.

Step 1: Measure one or more of the biomarkers in Table 1 and Table 2 and urinary creatinine pre and post surgery.

Step 2: For all measured biomarkers except urinary creatinine, divide the marker value by the value of urinary creatinine.

Step 3: Each of the processed marker measurements of markers in Table 1 are compared to marker-specific cutoffs. The number of markers that exceed the marker-specific cutoff will be determined If a pre-specified number of markers exceed the cutoff, the patient will be classified as belonging to the RIFLE I/F category. It may be that it is required that all markers exceed the cutoff, or all but one marker, or all but two markers etc. or only a single marker exceeds the cutoff. If the patient is classified as RIFLE I/F, the evaluation stops here, otherwise, the evaluation may proceed to the next step.

Step 4: Take measurement of a single marker in Table 2 or a weighted average of all processed marker measurements of markers in Table 2 and compare the result to a pre-specified cutoff. The weights used may be the same for all markers, however they may also be specific for each marker. If the weighted average is above the cutoff, classify the result as RIFLE R. If the patient is not classified as RIFLE R, go to the next step.

Step 5: Classify the patient as “No AKI”.

Method 3: Preprocessing and Baseline Normalization

Step 1: Measure one or more of the biomarkers in Table 1 and Table 2 and urinary creatinine pre and post surgery.

Step 2: For each biomarker, divide the value of the post-surgery sample by the value of the baseline sample. For each subsequent step, use these resulting values.

Step 3: Each of the processed marker measurements of markers in Table 1 are compared to marker-specific cutoffs. The number of markers that exceed the marker-specific cutoff will be determined. If a pre-specified number of markers exceed the cutoff, the patient will be classified as belonging to the RIFLE I/F category. It may be that it is required that all markers exceed the cutoff, or all but one marker, or all but two markers etc. or only a single marker exceeds the cutoff. If the patient is classified as RIFLE I/F, the evaluation stops here, otherwise, the evaluation may proceed to the next step.

Step 4: Take a measurement of a single marker or a weighted average of all processed marker measurements of markers in Table 2 and compare the result to a pre-specified cutoff. The weights used may be the same for all markers, however they may also be specific for each marker. If the weighted average is above the cutoff, classify the result as RIFLE R. If the patient is not classified as RIFLE R, go to the next step.

Step 5: Classify the patient as “No AKI”.

Method 4: Preprocessing, Urinary Creatinine and Baseline Normalization

Step 1: Measure any of the biomarkers in Table 1 and/or Table 2 including urinary creatinine, pre and post surgery.

Step 2: For each biomarker and the baseline as well as post-surgery samples, divide the value of the marker by the value of urinary creatinine in the same sample. Use the resulting values for the next step.

Step 3: For each biomarker, divide the value of the post-surgery sample by the value of the baseline sample. For each subsequent step, use these resulting values.

Step 4: Each of the processed marker measurements of markers in Table 1 are compared to marker-specific cutoffs. The number of markers that exceed the marker-specific cutoff will be determined. If a pre-specified number of markers exceed the cutoff, the patient will be classified as belonging to the RIFLE I/F category. It may be that it is required that all markers exceed the cutoff, or all but one marker, or all but two markers etc. or only a single marker exceeds the cutoff. If the patient is classified as RIFLE I/F, the evaluation stops here, otherwise, the evaluation proceeds at the next step.

Step 5: Take a weighted average of all processed marker measurements of markers in Table 2 and compare the result to a pre-specified cutoff. The weights used may be the same for all markers, however they may also be specific for each marker. If the weighted average is above the cutoff, classify the result as RIFLE R. If the patient is not classified as RIFLE R, go to the next step.

Step 6: Classify the patient as “No AKI”.

Additional Classification Methods:

Instead of the classification methods mentioned above for classifying patients as RIFLE I/F, RIFLE R or No AKI, a number of other standard classification tools could be used as well. Possible methods can be, but are not restricted to:
Linear regression, logistic regression, multinomial regression
Penalized linear or logistic or multinomial regression
Support Vector Machines
Linear Discriminant Analysis
Quadratic Discriminant Analysis
Classification and Regression Trees
Random Forests

These and other similar methods are all considered standard for people trained in the art and are readily applicable for any of the classification steps described above. For a more detailed reference to these and other methods see the “Elements of Statistical Learning” by Hastie, Tibshirani and Friedman.

To facilitate the sample analysis operation, data obtained may be analyzed using a digital computer. Typically, the computer will be appropriately programmed for receipt and storage of the data from the device, as well as for analysis and reporting of the data gathered, for example, subtraction of the background, verifying that controls have performed properly, normalizing the signals, interpreting fluorescence data to determine the amount of hybridized target, normalization of background, and the like.

Acute Kidney Treatments

For treatment of AKI, clinical examinations of novel therapeutic agents such as anti-apoptosis/anti-necrosis agents, anti-inflammatory agents, anti-septic agents, various growth factors, and vasodilator drugs may be used but results are less than satisfactory. The lack of satisfactory therapeutic agents for AKI is in particular because of lack of early biomarkers suitable for diagnosis of AKI thus making it near impossible to carry out early intervention.

There are many ways to treat AKI in the art, for example, treatment strategies include:
Change fluid management
Change treatment regimen (replace nephrotoxic drugs with other less nephrotoxic drugs, cease treatment with nephrotoxic drugs, change formulation of drugs to less nephrotoxic formulations)
Avoid treatments/clinical routine which may harm the kidney or worsen pre-existing kidney injury (e.g. angiography, administration of contrast dye)
Initiate renal replacement treatment or supportive care

Available Drugs, which are used to treat AKI:
Drugs that increase renal perfusion, e.g. Fenoldopam
Drug that inhibit inflammation and oxidative stress, e.g. N-Acetyl-Cystein
Diuretics, e.g. furosemide
Dopamine
Atrial natriuretic peptide
Recombinant human (rh)IGF-1
Theophylline

Drug candidates to treat AKI or proposed treatment strategies:
P38 inhibitor, e.g. Novartis BCT197
P53 inhibitor, e.g. Quark I5NP/Quark QPI-1002
Iron chelator, e.g. Deferiprone
Neutral endopeptidase (NEP) inhibitors and/or endothelin converting enzyme (ECE) inhibitors or dual inhibitorsActivators of the key receptors of the bone morphogenetic protein (BMP) family, e.g. THR-184
melanocortin (alpha-MSH) peptide analogues, such as ZP1480 (ABT-719) or AP214
Inhibitors of inflammatory pathways
Stem cell therapies

The method of the invention allows for the prediction of the severity of AKI based on determining the concentration of one or more markers present in Table 1 and/or Table 2. Accordingly, based on the results obtained using the method of the invention, physicians will be able to determine the best form of therapeutic intervention. The present invention can determine if an individual is likely to develop RIFLE I/F, RIFLE R or no AKI, which is crucial for selecting the appropriate therapeutic strategy for each patient individually. For example, if the subject is predicted to develop RIFLE I/F, the physician would likely treat with supporting renal function therapy such as dialysis but if the individual is predicted to develop RIFLE R, the subject would not be provided with dialysis. The present invention allows for the first time the prediction of what grade of severity of AKI an individual might have following cardiac surgery. Therefore this innovation is the basis for personalized therapies to treat or prevent AKI and thus will help to improve patient outcomes.

### EXAMPLES

### Example 1

### Overview of Clinical Data

The data for this analysis was collected in an observational, prospective, exploratory study in patients having cardiopulmonary bypass surgery. Patients with written consent aged 18 years or of any gender who underwent elective surgery could be included in the trial. Out of the patients enrolled in the trial, a patient had to meet the following criteria in order to be evaluable in our analysis:
The patient completed the study
Has a baseline/screening serum creatinine value as well as at least two serum creatinine measurements in the 24 to 72 hour time widow. As serum creatinine is commonly only taken once every 24 hours, for practical purposes we viewed this criterion as fulfilled if the serum creatinine were in the 12 hour to 84 hour window.
The patient has at least two collected urine samples at the 1, 2, 4 or 8 hour time points
The patient has at least one urine sample collected at the 12, 24 or 48 hour time points.

In the study, a total of 220 patients were enrolled, out of which 200 were evaluable according to the criteria above.

For the evaluable patients, we also assessed their AKI status. In order to be assessed as having AKI of one of the levels “Risk”, “Injury” or “Failure”, the change in a patient's serum creatinine from baseline has to be above the threshold for a period of at least 36 hours (to exclude transient rises of serum creatinine due to pre-renal azotemia). Furthermore, we are only counting a patient as having an AKI if the criterion in met within the first 7 days after the surgery (as an AKI caused by the CPB surgery should have presented by that time). We introduced the 36 hour time window so that patients who only have a very brief increase in serum creatinine do not count as AKI cases. We believe that such a sustained increase of serum creatinine gives a much better evaluation of permanent injury to the kidney. In particular, the classification was according to the following rules:
If a patient has an increase of more than 200% above baseline serum creatinine level for a time period of at least 36 hours, the patient is classified as “Failure”.
If a patient is not classified as “Failure” and has an increase of serum creatinine of at least 100% above baseline for at least 36 hours, the patient is classified as “Injury”.
If a patient is not classified as “Injury” or “Failure” and has an increase of serum creatinine of at least 50% above baseline for a time period of at least 36 hours, the patient is classified as “Risk”.
If a patient is not classified as “Risk”, “Injury” or “Failure”, the patient is classified as “No AKI”.

These criteria have to be met within 7 days after CPB surgery. As the baseline value of serum creatinine we use the average of the screening and pre-op values if both are available, the screening value if the pre-op value is missing and the pre-op value if the screening value is missing. A patient were both the screening and pre-op serum creatinine value are missing is considered as not evaluable. Kits for determining the biomarker levels were obtained from Rules Based Medicine (RBM) using the KidneyMAP® kits.

Among the 200 patients, we have according to these criteria 187 patients classified as “No AKI”, 8 as “Risk”, 3 as “Injury” and 2 as “Failure”. A table of summary statistics of key clinical variables is provided in the table below.

TABLE 5
Summary table of clinical variables grouped by severity of AKI. For discrete variables,
percentages in the group are given in paratheses. For continuous variables, standard deviation
is given in parantheses.

| | [ALL] | NoAKI | Risk | Injury | Failure | |
|---|---|---|---|---|---|---|
| | N = 200 | N = 187 | N = 8 | N = 3 | N = 2 | p. overall |
| Age | 64.9 (10.8) | 64.2 (10.6) | 75.8 (6.96) | 69.0 (12.3) | 81.0 (8.49) | 0.003 |
| Gender: | | | | | | 0.468 |
| FEMALE | 53 (26.5%) | 48 (25.7%) | 3 (37.5%) | 1 (33.3%) | 1 (50.0%) | |
| MALE | 147 (73.5%) | 139 (74.3%) | 5 (62.5%) | 2 (66.7%) | 1 (50.0%) | |
| Race: | | | | | | 1.000 |
| CAUCASIAN | 199 (99.5%) | 186 (99.5%) | 8 (100%) | 3 (100%) | 2 (100%) | |
| OTHER | 1 (0.50%) | 1 (0.53%) | 0 (0.00%) | 0 (0.00%) | 0 (0.00%) | |
| Baseline eGFR category: | | | | | | 0.063 |
| 30-60 | 62 (31.0%) | 54 (28.9%) | 4 (50.0%) | 2 (66.7%) | 2 (100%) | |
| 60-90 | 79 (39.5%) | 74 (39.6%) | 4 (50.0%) | 1 (33.3%) | 0 (0.00%) | |
| >=90 | 59 (29.5%) | 59 (31.6%) | 0 (0.00%) | 0 (0.00%) | 0 (0.00%) | |
| Height | 168 (8.87) | 168 (8.85) | 164 (6.76) | 165 (11.7) | 163 (18.4) | 0.504 |
| Weight | 78.9 (15.5) | 79.4 (15.7) | 71.2 (12.0) | 73.7 (6.03) | 72.0 (1.41) | 0.405 |
| Body Mass Index | 27.9 (4.88) | 28.0 (4.94) | 26.4 (4.27) | 27.1 (2.71) | 27.6 (5.66) | 0.820 |
| Length of surgery in hours | 3.69 (0.99) | 3.69 (0.97) | 3.34 (1.07) | 3.27 (0.37) | 5.61 (1.52) | 0.027 |
| Time on bypass in hours | 1.69 (0.73) | 1.66 (0.70) | 1.86 (0.63) | 1.70 (0.53) | 3.87 (1.79) | <0.001 |

### Example 2

### Biomarkers Under Consideration and Preprocessing

For each biomarker we performed certain pre-processing steps before using them in the analysis. Due to the sensitivity of the assay used, it can happen that a marker in urine is below the limit of detection and therefore no value reported or below the limit of quantitation (for which a value may be reported). In both these cases, we replace the measured value with a value equal to half the limit of quantitation for this biomarker and sample lot. The resulting measurement is in the follow referred to as the pre-processed measurement.

In our following analysis, we use this pre-processed measurement as well as a urinary creatinine (UCREA) normalized-measurement. For this normalization, the pre-processed measurement of urinary creatinine from the same urine sample is being used. The normalization is being performed by dividing the pre-processed biomarker measurement in urine by the pre-processed urinary creatinine measurement from the same urine sample. We refer to this in the following as the UCREA-normalized biomarker measurement.

In addition to the pre-processed and UCREA-normalized measurements, we also evaluate the change of the pre-processed and UCREA-normalized measurements from baseline. For this, a pre-op urine sample has to be available for the patient. If the pre-op urine sample is missing, the change from baseline measurement is considered missing for this patient. For a pre-processed biomarker for a patient, in order to obtain the fold-change from baseline, the pre-processed biomarker measurement is divided by the pre-processed baseline measurement for the same patient. For a UCREA-normalized biomarker for a patient, in order to obtain the fold-change from baseline, the UCREA-normalized biomarker measurement is divided by the normalized baseline measurement for the same patient.

All in all, we consider in our analysis the pre-processed, normalized, pre-processed fold-change from baseline and UCREA-normalized fold-change from baseline measurements for all biomarkers. To each of these 4 derived variables we apply a logarithmic transformation to base 10 before use.

### Example 3

### Univariate Assessment of Models

For each biomarker under consideration, we calculate an Area under the Receiver Operating curve (AUC) with respect to two binary endpoints. In the first assessment, we compare patients classified as “Injury” or “Failure” against patients classified as “No AKI” or Risk”. In the second assessment, we exclude patients classified as either “Injury” or “Failure” and only compare patients classified as “Risk” against patients classified as “No AKI”.

### Example 4

### Classifying “Injury” or “Failure” Against “Risk” or “No AKI”

When classifying patients as “Injury” or “Failure” against “Risk” or “No AKI”, then the biomarkers Alpha-1-microglobulin (A1Micro), Clusterin (CLU), Cystatin-C (CYSC), Interleukin-18 (IL-18), Neutrophil gelatinase-associated lipocalin (NGAL) and Trefoil-factor 3 (TFF3) show performance in the time range from 0 to 48 hours using the pre-processed, UCREA-normalized, pre-processed fold-change from baseline and UCREA-normalized fold-change from baseline measurements.

In the following tables, we will present data for each of these biomarkers, for each of the 4 transformations and for each of the time points 0, 1, 2, 4, 8, 12, 24 and 48 hours after arrival at ICU.

TABLE 6
AUCs for pre-processed biomarkers classifying “Injury”
or “Failure” against “Risk” or “No AKI”. Time points
up to up to 48 hours after arrival at ICU are included and
confidence intervals for the AUCs are given as well.

| | Timepoint | | | | |
|---|---|---|---|---|---|
| Biomarker | (hours) | Cases | Controls | AUC | CI(AUC) |
| A1Micro | 0 | 5 | 183 | 75.25 | (63.55, 85.58) |
| A1Micro | 1 | 4 | 192 | 84.44 | (71.94, 95.71) |
| A1Micro | 2 | 5 | 193 | 87.72 | (80, 94.4) |
| A1Micro | 4 | 5 | 193 | 88.86 | (82.23, 94.72) |
| A1Micro | 8 | 5 | 188 | 84.31 | (72.92, 93.35) |
| A1Micro | 12 | 4 | 188 | 71.28 | (50.4, 91.49) |
| A1Micro | 24 | 5 | 187 | 68.13 | (55.08, 81.5) |
| A1Micro | 48 | 3 | 180 | 81.76 | (63.89, 97.22) |
| CLU | 0 | 5 | 183 | 73.33 | (55.68, 90.93) |
| CLU | 1 | 4 | 192 | 74.48 | (50.78, 96.61) |
| CLU | 2 | 5 | 193 | 73.78 | (53.32, 93.16) |
| CLU | 4 | 5 | 193 | 67.25 | (36.16, 96.48) |
| CLU | 8 | 5 | 188 | 69.41 | (47.39, 89.26) |
| CLU | 12 | 4 | 188 | 72.41 | (29.52, 94.95) |
| CLU | 24 | 5 | 187 | 47.59 | (15.61, 80.91) |
| CLU | 48 | 3 | 180 | 60.74 | (47.41, 77.22) |
| CYSC | 0 | 5 | 183 | 84.1 | (65.08, 97.6) |
| CYSC | 1 | 4 | 192 | 81.58 | (51.76, 98.31) |
| CYSC | 2 | 5 | 193 | 84.92 | (63.37, 98.13) |
| CYSC | 4 | 5 | 193 | 85.23 | (59.17, 99.17) |
| CYSC | 8 | 5 | 188 | 57.07 | (25.53, 84.68) |
| CYSC | 12 | 4 | 188 | 56.58 | (21, 93.42) |
| CYSC | 24 | 5 | 187 | 53.16 | (18.77, 87.75) |
| CYSC | 48 | 3 | 180 | 95.93 | (90, 100) |
| IL-18 | 0 | 5 | 183 | 74.64 | (49.83, 98.8) |
| IL-18 | 1 | 4 | 192 | 84.18 | (53.12, 100) |
| IL-18 | 2 | 5 | 193 | 73.99 | (47.15, 100) |
| IL-18 | 4 | 5 | 193 | 83.83 | (56.37, 99.48) |
| IL-18 | 8 | 5 | 188 | 56.79 | (27.75, 83.8) |
| IL-18 | 12 | 4 | 188 | 68.35 | (34.44, 90.3) |
| IL-18 | 24 | 5 | 187 | 58.18 | (41.6, 75.46) |
| IL-18 | 48 | 3 | 180 | 52.78 | (43.89, 61.39) |
| NGAL | 0 | 5 | 183 | 70.82 | (38.58, 94.76) |
| NGAL | 1 | 4 | 192 | 80.99 | (51.69, 97.66) |
| NGAL | 2 | 5 | 193 | 82.33 | (64.51, 97.51) |
| NGAL | 4 | 5 | 193 | 84.3 | (66.53, 97.41) |
| NGAL | 8 | 5 | 188 | 80.48 | (70.32, 90.21) |
| NGAL | 12 | 4 | 188 | 78.32 | (61.63, 91.49) |
| NGAL | 24 | 5 | 187 | 75.4 | (57.81, 90.91) |
| NGAL | 48 | 3 | 180 | 78.52 | (59.17, 92.41) |
| TFF3 | 0 | 5 | 183 | 74.26 | (60.27, 86.01) |
| TFF3 | 1 | 4 | 192 | 86.26 | (75.65, 94.4) |
| TFF3 | 2 | 5 | 193 | 86.27 | (79.84, 91.97) |
| TFF3 | 4 | 5 | 193 | 74.3 | (54.66, 93.47) |
| TFF3 | 8 | 5 | 188 | 66.6 | (51.96, 80.22) |
| TFF3 | 12 | 4 | 188 | 57.58 | (41.88, 75.27) |
| TFF3 | 24 | 5 | 187 | 62.89 | (48.02, 77.86) |
| TFF3 | 48 | 3 | 180 | 69.91 | (49.44, 92.79) |

For the biomarkers A1Micro, CLU, CYSC, IL-18, NGAL and TFF3 using the pre-processing transformation, it can be seen in Table that for the timepoints 0, 1, 2, 4, 8, 12, 24 and 48 hours these markers can be used to distinguish patients with AKI classified as “Injury” or “Failure” from those classified as “Risk” or “No AKI”. For all these markers, the timepoints 1 hour, 2 hours, 4 hours, 8 hours and 48 hours show especially good performance. Also, the markers A1Micro, CYSC, IL-18, NGAL and TFF3 are particularly good for classifying severe cases of AKI in this instance.

TABLE 7
AUCs for normalized biomarkers classifying “Injury”
or “Failure” against “Risk” or “No AKI”. Time points
up to up to 48 hours after arrival at ICU are included
and confidence intervals for the AUCs are given as well.

| | Timepoint | | | | |
|---|---|---|---|---|---|
| Biomarker | (hours) | Cases | Controls | AUC | CI(AUC) |
| A1Micro | 0 | 5 | 183 | 79.13 | (58.8, 93.99) |
| A1Micro | 1 | 4 | 192 | 85.03 | (66.79, 96.74) |
| A1Micro | 2 | 5 | 193 | 89.95 | (80.41, 96.89) |
| A1Micro | 4 | 5 | 193 | 91.92 | (80.83, 98.76) |
| A1Micro | 8 | 5 | 188 | 89.68 | (75.32, 98.62) |
| A1Micro | 12 | 4 | 188 | 87.23 | (77.13, 96.28) |
| A1Micro | 24 | 5 | 187 | 83.42 | (69.52, 94.97) |
| A1Micro | 48 | 3 | 180 | 97.41 | (94.07, 99.63) |
| CLU | 0 | 5 | 183 | 80 | (61.64, 95.3) |
| CLU | 1 | 4 | 192 | 74.54 | (40.36, 96.61) |
| CLU | 2 | 5 | 193 | 76.27 | (51.4, 96.99) |
| CLU | 4 | 5 | 193 | 76.68 | (43.73, 98.45) |
| CLU | 8 | 5 | 188 | 57.18 | (26.91, 81.38) |
| CLU | 12 | 4 | 188 | 65.89 | (23.54, 94.15) |
| CLU | 24 | 5 | 187 | 51.98 | (16.89, 88.03) |
| CLU | 48 | 3 | 180 | 79.81 | (65, 94.46) |
| CYSC | 0 | 5 | 183 | 83.06 | (58.91, 98.25) |
| CYSC | 1 | 4 | 192 | 79.56 | (44.27, 98.7) |
| CYSC | 2 | 5 | 193 | 82.38 | (53.78, 98.55) |
| CYSC | 4 | 5 | 193 | 81.66 | (46.01, 100) |
| CYSC | 8 | 5 | 188 | 50.69 | (25.32, 70.21) |
| CYSC | 12 | 4 | 188 | 60.97 | (20.74, 88.03) |
| CYSC | 24 | 5 | 187 | 53.69 | (16.04, 90.91) |
| CYSC | 48 | 3 | 180 | 97.41 | (93.33, 100) |
| IL-18 | 0 | 5 | 183 | 72.73 | (38.3, 99.78) |
| IL-18 | 1 | 4 | 192 | 79.82 | (38.87, 100) |
| IL-18 | 2 | 5 | 193 | 74.92 | (39.17, 100) |
| IL-18 | 4 | 5 | 193 | 80.21 | (41.55, 100) |
| IL-18 | 8 | 5 | 188 | 68.77 | (30.48, 98.07) |
| IL-18 | 12 | 4 | 188 | 81.32 | (62.5, 99.47) |
| IL-18 | 24 | 5 | 187 | 58.18 | (28.77, 86.31) |
| IL-18 | 48 | 3 | 180 | 82.41 | (68.33, 92.78) |
| NGAL | 0 | 5 | 183 | 71.48 | (34.1, 96.07) |
| NGAL | 1 | 4 | 192 | 78.91 | (44.65, 97.92) |
| NGAL | 2 | 5 | 193 | 82.59 | (60, 97.62) |
| NGAL | 4 | 5 | 193 | 83.63 | (54.82, 99.17) |
| NGAL | 8 | 5 | 188 | 83.62 | (59.68, 98.19) |
| NGAL | 12 | 4 | 188 | 86.84 | (70.48, 99.2) |
| NGAL | 24 | 5 | 187 | 88.72 | (80.91, 95.83) |
| NGAL | 48 | 3 | 180 | 94.26 | (89.44, 98.33) |
| TFF3 | 0 | 5 | 183 | 80.93 | (53.44, 97.7) |
| TFF3 | 1 | 4 | 192 | 88.15 | (69.79, 99.48) |
| TFF3 | 2 | 5 | 193 | 92.44 | (78.55, 99.79) |
| TFF3 | 4 | 5 | 193 | 90.98 | (76.47, 99.38) |
| TFF3 | 8 | 5 | 188 | 87.18 | (68.93, 97.71) |
| TFF3 | 12 | 4 | 188 | 84.57 | (67.95, 96.81) |
| TFF3 | 24 | 5 | 187 | 78.18 | (60.43, 94.97) |
| TFF3 | 48 | 3 | 180 | 94.44 | (87.22, 99.44) |

For the biomarkers A1Micro, CLU, CYSC, IL-18, NGAL and TFF3 using the UCREA-normalization transformation, it can be seen in Table that for the timepoints 0, 1, 2, 4, 8, 12, 24 and 48 hours these markers can be used to distinguish patients with AKI classified as “Injury” or “Failure” from those classified as “Risk” or “No AKI”. For all these markers, the timepoints 1 hour, 2 hours, 4 hours, 8 hours and 48 hours show especially good performance. Also, the markers A1Micro, CYSC, IL-18, NGAL and TFF3 are particularly good for classifying severe cases of AKI in this instance.

TABLE 8
AUCs for pre-processed change from baseline biomarkers
classifying “Injury” or “Failure” against “Risk” or “No AKI”.
Time points up to up to 48 hours after arrival at ICU are included
and confidence intervals for the AUCs are given as well.

| | Timepoint | | | | |
|---|---|---|---|---|---|
| Biomarker | (hours) | Cases | Controls | AUC | CI(AUC) |
| A1Micro | 0 | 5 | 183 | 44.64 | (18.03, 72.35) |
| A1Micro | 1 | 4 | 192 | 73.83 | (56.51, 91.15) |
| A1Micro | 2 | 5 | 193 | 63.63 | (31.09, 89.64) |
| A1Micro | 4 | 5 | 193 | 60.83 | (34.92, 84.87) |
| A1Micro | 8 | 5 | 188 | 57.87 | (28.4, 83.51) |
| A1Micro | 12 | 4 | 188 | 59.71 | (32.18, 88.04) |
| A1Micro | 24 | 5 | 187 | 53.9 | (27.37, 77.76) |
| A1Micro | 48 | 3 | 180 | 81.85 | (73.14, 90.56) |
| CLU | 0 | 5 | 183 | 71.91 | (40.22, 97.81) |
| CLU | 1 | 4 | 192 | 81.9 | (50.39, 98.96) |
| CLU | 2 | 5 | 193 | 72.85 | (46.42, 96.89) |
| CLU | 4 | 5 | 193 | 75.44 | (51.61, 94.2) |
| CLU | 8 | 5 | 188 | 58.35 | (29.63, 87.45) |
| CLU | 12 | 4 | 188 | 52.86 | (16.88, 89.1) |
| CLU | 24 | 5 | 187 | 49.04 | (24.81, 72.19) |
| CLU | 48 | 3 | 180 | 79.35 | (64.07, 97.22) |
| CYSC | 0 | 5 | 183 | 73.88 | (41.31, 99.56) |
| CYSC | 1 | 4 | 192 | 83.07 | (54.56, 99.48) |
| CYSC | 2 | 5 | 193 | 73.06 | (45.59, 98.34) |
| CYSC | 4 | 5 | 193 | 76.99 | (51.6, 99.59) |
| CYSC | 8 | 5 | 188 | 58.03 | (25.8, 85.91) |
| CYSC | 12 | 4 | 188 | 58.71 | (34.04, 83.92) |
| CYSC | 24 | 5 | 187 | 56.63 | (30.53, 78.93) |
| CYSC | 48 | 3 | 180 | 96.48 | (90.56, 100) |
| IL-18 | 0 | 5 | 183 | 74.04 | (44.86, 97.7) |
| IL-18 | 1 | 4 | 192 | 88.54 | (67.45, 100) |
| IL-18 | 2 | 5 | 193 | 75.28 | (44.09, 99.79) |
| IL-18 | 4 | 5 | 193 | 85.85 | (69.32, 99.69) |
| IL-18 | 8 | 5 | 188 | 60.21 | (32.41, 84.97) |
| IL-18 | 12 | 4 | 188 | 65.89 | (40.16, 90.56) |
| IL-18 | 24 | 5 | 187 | 59.52 | (40.32, 77.76) |
| IL-18 | 48 | 3 | 180 | 69.44 | (62.03, 76.39) |
| NGAL | 0 | 5 | 183 | 54.97 | (23.28, 87.76) |
| NGAL | 1 | 4 | 192 | 76.3 | (53.5, 95.7) |
| NGAL | 2 | 5 | 193 | 69.95 | (40.82, 92.33) |
| NGAL | 4 | 5 | 193 | 69.22 | (52.02, 86.84) |
| NGAL | 8 | 5 | 188 | 44.57 | (20, 68.72) |
| NGAL | 12 | 4 | 188 | 53.72 | (15.95, 92.83) |
| NGAL | 24 | 5 | 187 | 49.95 | (20.32, 80.11) |
| NGAL | 48 | 3 | 180 | 74.63 | (57.78, 89.44) |
| TFF3 | 0 | 5 | 183 | 57.81 | (30.6, 82.19) |
| TFF3 | 1 | 4 | 192 | 66.28 | (50.65, 86.33) |
| TFF3 | 2 | 5 | 193 | 54.15 | (25.38, 82.28) |
| TFF3 | 4 | 5 | 193 | 58.86 | (25.8, 89.23) |
| TFF3 | 8 | 5 | 188 | 60.37 | (35.74, 82.98) |
| TFF3 | 12 | 4 | 188 | 54.52 | (26.46, 82.45) |
| TFF3 | 24 | 5 | 187 | 61.34 | (29.52, 86.63) |
| TFF3 | 48 | 3 | 180 | 67.04 | (57.22, 75.93) |

For the biomarkers A1Micro, CLU, CYSC, IL-18, NGAL and TFF3 using the pre-processed fold-change from baseline transformation, it can be seen in Table that for the timepoints 0, 1, 2, 4, 8, 12, 24 and 48 hours these markers can be used to distinguish patients with AKI classified as “Injury” or “Failure” from those classified as “Risk” or “No AKI”. For all these markers, the timepoints 1 hour, 2 hours, 4 hours and 48 hours show especially good performance. Also, the markers CLU, CYSC, IL-18 and NGAL are particularly good for classifying severe cases of AKI in this instance.

TABLE 9
AUCs for normalized change from baseline biomarkers
classifying “Injury” or “Failure” against “Risk” or “No AKI”.
Time points up to up to 48 hours after arrival at ICU are included
and confidence intervals for the AUCs are given as well.

| | Timepoint | | | | |
|---|---|---|---|---|---|
| Biomarker | (hours) | Cases | Controls | AUC | CI(AUC) |
| A1Micro | 0 | 5 | 183 | 48.42 | (18.9, 77.6) |
| A1Micro | 1 | 4 | 192 | 72.01 | (53.25, 90.63) |
| A1Micro | 2 | 5 | 193 | 64.77 | (36.37, 87.77) |
| A1Micro | 4 | 5 | 193 | 72.02 | (47.15, 92.64) |
| A1Micro | 8 | 5 | 188 | 62.55 | (34.26, 87.66) |
| A1Micro | 12 | 4 | 188 | 64.1 | (40.56, 89.49) |
| A1Micro | 24 | 5 | 187 | 55.08 | (27.17, 78.93) |
| A1Micro | 48 | 3 | 180 | 89.07 | (76.67, 98.7) |
| CLU | 0 | 5 | 183 | 69.84 | (37.59, 98.25) |
| CLU | 1 | 4 | 192 | 77.86 | (39.32, 98.96) |
| CLU | 2 | 5 | 193 | 74.3 | (46.53, 98.13) |
| CLU | 4 | 5 | 193 | 79.07 | (45.8, 98.76) |
| CLU | 8 | 5 | 188 | 55.43 | (21.7, 86.6) |
| CLU | 12 | 4 | 188 | 50 | (10.63, 90.16) |
| CLU | 24 | 5 | 187 | 55.94 | (27.06, 78.61) |
| CLU | 48 | 3 | 180 | 94.26 | (89.44, 98.33) |
| CYSC | 0 | 5 | 183 | 70.16 | (39.89, 98.91) |
| CYSC | 1 | 4 | 192 | 78.12 | (37.5, 99.35) |
| CYSC | 2 | 5 | 193 | 72.33 | (43.21, 98.45) |
| CYSC | 4 | 5 | 193 | 78.03 | (45.28, 99.59) |
| CYSC | 8 | 5 | 188 | 56.7 | (22.01, 90.32) |
| CYSC | 12 | 4 | 188 | 57.45 | (26.06, 89.37) |
| CYSC | 24 | 5 | 187 | 52.73 | (21.17, 81.71) |
| CYSC | 48 | 3 | 180 | 99.26 | (97.41, 100) |
| IL-18 | 0 | 5 | 183 | 73.44 | (44.92, 97.93) |
| IL-18 | 1 | 4 | 192 | 85.94 | (58.59, 100) |
| IL-18 | 2 | 5 | 193 | 77.62 | (51.71, 99.59) |
| IL-18 | 4 | 5 | 193 | 82.38 | (49.12, 100) |
| IL-18 | 8 | 5 | 188 | 66.1 | (34.43, 94.22) |
| IL-18 | 12 | 4 | 188 | 70.74 | (48.67, 93.09) |
| IL-18 | 24 | 5 | 187 | 56.04 | (26.95, 85.99) |
| IL-18 | 48 | 3 | 180 | 77.04 | (42.22, 97.22) |
| NGAL | 0 | 5 | 183 | 56.61 | (22.18, 87.32) |
| NGAL | 1 | 4 | 192 | 75.65 | (47.26, 94.79) |
| NGAL | 2 | 5 | 193 | 72.02 | (50.67, 93.58) |
| NGAL | 4 | 5 | 193 | 77.31 | (53.37, 94.92) |
| NGAL | 8 | 5 | 188 | 62.13 | (46.91, 82.87) |
| NGAL | 12 | 4 | 188 | 54.12 | (20.21, 86.7) |
| NGAL | 24 | 5 | 187 | 46.42 | (16.58, 73.91) |
| NGAL | 48 | 3 | 180 | 72.59 | (26.11, 99.44) |
| TFF3 | 0 | 5 | 183 | 65.14 | (33, 88.52) |
| TFF3 | 1 | 4 | 192 | 46.22 | (16.4, 69.93) |
| TFF3 | 2 | 5 | 193 | 50.98 | (22.27, 77) |
| TFF3 | 4 | 5 | 193 | 51.81 | (24.97, 76.17) |
| TFF3 | 8 | 5 | 188 | 60.11 | (27.02, 85.64) |
| TFF3 | 12 | 4 | 188 | 56.91 | (21.81, 86.17) |
| TFF3 | 24 | 5 | 187 | 65.88 | (31.44, 91.44) |
| TFF3 | 48 | 3 | 180 | 72.59 | (34.44, 98.33) |

For the biomarkers A1Micro, CLU, CYSC, IL-18, NGAL and TFF3 using the UCREA-normalization fold-change from baseline transformation, it can be seen in Table that for the timepoints 0, 1, 2, 4, 8, 12, 24 and 48 hours these markers can be used to distinguish patients with AKI classified as “Injury” or “Failure” from those classified as “Risk” or “No AKI”. For all these markers, the timepoints 1 hour, 2 hours, 4 hours and 48 hours show especially good performance. Also, the markers CLU, CYSC, IL-18 and NGAL are particularly good for classifying severe cases of AKI in this instance.

### Example 5

### Classifying “Risk” Against “No AKI”

When comparing patients with AKI classified as “Risk” against patients classified as “No AKI”, the biomarkers A1Micro, B2Micro and TFF3 show performance in the time range from 0 to 48 hours using the pre-processing, UCREA-normalization, pre-processing fold-change from baseline, UCREA-normalization fold-change from baseline transformations.

In the following tables, we will present data for each of these biomarkers, transformations and time point 0, 1, 2, 4, 8, 12, 24 and 48 hours.

TABLE 10
AUCs for pre-processed biomarkers classifying “Risk”
against “No AKI”. Time points up to up to 48 hours
after arrival at ICU are included and confidence intervals
for the AUCs are given as well.

| | Timepoint | | | | |
|---|---|---|---|---|---|
| Biomarker | (hours) | Cases | Controls | AUC | CI(AUC) |
| A1Micro | 0 | 6 | 177 | 57.02 | (39.5, 72.93) |
| A1Micro | 1 | 8 | 184 | 64.91 | (51.73, 76.32) |
| A1Micro | 2 | 8 | 185 | 56.82 | (37.03, 74.06) |
| A1Micro | 4 | 8 | 185 | 69.26 | (52.6, 82.43) |
| A1Micro | 8 | 8 | 180 | 62.57 | (35.76, 85.22) |
| A1Micro | 12 | 7 | 181 | 58.01 | (35.75, 79.48) |
| A1Micro | 24 | 7 | 180 | 51.63 | (37.06, 65.84) |
| A1Micro | 48 | 7 | 173 | 45.62 | (24.44, 66.6) |
| B2Micro | 0 | 6 | 177 | 57.3 | (32.48, 80.65) |
| B2Micro | 1 | 8 | 184 | 65.15 | (43.07, 84.95) |
| B2Micro | 2 | 8 | 185 | 59.16 | (34.59, 83.04) |
| B2Micro | 4 | 8 | 185 | 71.25 | (51.32, 88.86) |
| B2Micro | 8 | 8 | 180 | 60.42 | (36.42, 81.18) |
| B2Micro | 12 | 7 | 181 | 48.42 | (32.2, 62.98) |
| B2Micro | 24 | 7 | 180 | 61.27 | (46.98, 75.16) |
| B2Micro | 48 | 7 | 173 | 65.48 | (50.7, 79.07) |
| TFF3 | 0 | 6 | 177 | 60.31 | (43.21, 77.97) |
| TFF3 | 1 | 8 | 184 | 69.6 | (58.29, 80.2) |
| TFF3 | 2 | 8 | 185 | 53.95 | (35.88, 71.08) |
| TFF3 | 4 | 8 | 185 | 66.15 | (53.88, 78.04) |
| TFF3 | 8 | 8 | 180 | 55.69 | (32.56, 76.5) |
| TFF3 | 12 | 7 | 181 | 55.92 | (32.39, 78.26) |
| TFF3 | 24 | 7 | 180 | 50.67 | (36.27, 65.08) |
| TFF3 | 48 | 7 | 173 | 60.65 | (36.54, 82.21) |

The biomarkers A1Micro, B2Micro and TFF3 using the pre-processing transformation show performance for classifying “Risk” against “No AKI” patients.

TABLE 11
AUCs for normalized biomarkers classifying “Risk” against
“No AM”. Time pointsup to up to 48 hours after arrival at
ICU are included and confidence intervals for the AUCs
are given as well.

| | Timepoint | | | | |
|---|---|---|---|---|---|
| Biomarker | (hours) | Cases | Controls | AUC | CI(AUC) |
| A1Micro | 0 | 6 | 177 | 59.23 | (40.72, 78.11) |
| A1Micro | 1 | 8 | 184 | 72.69 | (60.66, 84.44) |
| A1Micro | 2 | 8 | 185 | 69.7 | (47.36, 86.93) |
| A1Micro | 4 | 8 | 185 | 77.87 | (59.56, 90.07) |
| A1Micro | 8 | 8 | 180 | 66.67 | (41.04, 89.79) |
| A1Micro | 12 | 7 | 181 | 71.27 | (51.74, 86.82) |
| A1Micro | 24 | 7 | 180 | 58.81 | (40.11, 76.19) |
| A1Micro | 48 | 7 | 173 | 51.94 | (26.91, 73.25) |
| B2Micro | 0 | 6 | 177 | 58.66 | (34.93, 82.68) |
| B2Micro | 1 | 8 | 184 | 66.51 | (43, 85.67) |
| B2Micro | 2 | 8 | 185 | 61.89 | (38.51, 84.67) |
| B2Micro | 4 | 8 | 185 | 73.31 | (54.46, 89.6) |
| B2Micro | 8 | 8 | 180 | 67.15 | (46.18, 84.86) |
| B2Micro | 12 | 7 | 181 | 56.08 | (38.04, 72.3) |
| B2Micro | 24 | 7 | 180 | 60.36 | (42.42, 76.59) |
| B2Micro | 48 | 7 | 173 | 65.52 | (50.54, 80.02) |
| TFF3 | 0 | 6 | 177 | 63.84 | (39.54, 87.71) |
| TFF3 | 1 | 8 | 184 | 78.12 | (61.07, 91.99) |
| TFF3 | 2 | 8 | 185 | 69.46 | (47.09, 88.11) |
| TFF3 | 4 | 8 | 185 | 79.53 | (68.51, 89.6) |
| TFF3 | 8 | 8 | 180 | 72.5 | (49.51, 90.42) |
| TFF3 | 12 | 7 | 181 | 73.95 | (61.96, 84.45) |
| TFF3 | 24 | 7 | 180 | 57.38 | (39.52, 71.9) |
| TFF3 | 48 | 7 | 173 | 66.76 | (41.45, 86.17) |

The biomarkers A1Micro, B2Micro and TFF3 using the UCREA-normalization transformation show performance for classifying “Risk” against “No AKI”. The performance of the markers is especially good at the 1, 2 and 4 hour time points.

TABLE 12
AUCs for pre-processed change from baseline biomarkers
classifying “Risk” against “No AKI”. Time points up to
up to 48 hours after arrival at ICU are included and
confidence intervals for the AUCs are given as well.

| | Timepoint | | | | |
|---|---|---|---|---|---|
| Biomarker | (hours) | Cases | Controls | AUC | CI(AUC) |
| A1Micro | 0 | 6 | 177 | 52.12 | (32.39, 70.34) |
| A1Micro | 1 | 8 | 184 | 57 | (41.85, 69.7) |
| A1Micro | 2 | 8 | 185 | 48.24 | (29.73, 65.27) |
| A1Micro | 4 | 8 | 185 | 57.4 | (39.12, 74.12) |
| A1Micro | 8 | 8 | 180 | 54.51 | (30.83, 76.94) |
| A1Micro | 12 | 7 | 181 | 51.7 | (32.2, 70.33) |
| A1Micro | 24 | 7 | 180 | 56.94 | (42.54, 71.47) |
| A1Micro | 48 | 7 | 173 | 62.84 | (44.1, 81.42) |
| B2Micro | 0 | 6 | 177 | 57.44 | (36.35, 76.46) |
| B2Micro | 1 | 8 | 184 | 55.91 | (36, 75.27) |
| B2Micro | 2 | 8 | 185 | 51.28 | (32.63, 68.11) |
| B2Micro | 4 | 8 | 185 | 62.09 | (43.58, 81.22) |
| B2Micro | 8 | 8 | 180 | 51.6 | (30.55, 72.5) |
| B2Micro | 12 | 7 | 181 | 60.3 | (40.17, 80.27) |
| B2Micro | 24 | 7 | 180 | 74.56 | (59.24, 87.9) |
| B2Micro | 48 | 7 | 173 | 70.11 | (52.93, 86.38) |
| TFF3 | 0 | 6 | 177 | 70.43 | (62.71, 77.59) |
| TFF3 | 1 | 8 | 184 | 68.44 | (54.69, 79.28) |
| TFF3 | 2 | 8 | 185 | 56.15 | (41.59, 69.5) |
| TFF3 | 4 | 8 | 185 | 67.2 | (51.08, 80.88) |
| TFF3 | 8 | 8 | 180 | 54.27 | (31.25, 75.87) |
| TFF3 | 12 | 7 | 181 | 51.85 | (30.46, 72.69) |
| TFF3 | 24 | 7 | 180 | 50.52 | (33.41, 66.55) |
| TFF3 | 48 | 7 | 173 | 61.85 | (40.05, 82.33) |

The biomarkers A1Micro, B2Micro and TFF3 using the UCREA-normalization fold-change from baseline shows performance for distinguishing patients classified as “Risk” from patients classified as “No AKI”.

TABLE 13
AUCs for normalized change from baseline biomarkers
classifying “Risk” against “No AKI”. Time points up to
up to 48 hours after arrival at ICU are included and
confidence intervals for the AUCs are given as well.

| | Timepoint | | | | |
|---|---|---|---|---|---|
| Biomarker | (hours) | Cases | Controls | AUC | CI(AUC) |
| A1Micro | 0 | 6 | 177 | 58 | (34.46, 79.1) |
| A1Micro | 1 | 8 | 184 | 53.8 | (35.33, 71.47) |
| A1Micro | 2 | 8 | 185 | 60 | (37.43, 79.19) |
| A1Micro | 4 | 8 | 185 | 50.2 | (29.72, 69.46) |
| A1Micro | 8 | 8 | 180 | 48.26 | (22.91, 72.64) |
| A1Micro | 12 | 7 | 181 | 55.01 | (32.12, 76.25) |
| A1Micro | 24 | 7 | 180 | 67.14 | (44.68, 86.35) |
| A1Micro | 48 | 7 | 173 | 78.2 | (61.93, 91.33) |
| B2Micro | 0 | 6 | 177 | 52.73 | (30.6, 72.88) |
| B2Micro | 1 | 8 | 184 | 51.97 | (27.85, 73.64) |
| B2Micro | 2 | 8 | 185 | 53.31 | (32.77, 72.64) |
| B2Micro | 4 | 8 | 185 | 57.57 | (39.53, 77.37) |
| B2Micro | 8 | 8 | 180 | 50 | (27.36, 73.06) |
| B2Micro | 12 | 7 | 181 | 60.69 | (41.91, 80.51) |
| B2Micro | 24 | 7 | 180 | 78.33 | (60.87, 92.78) |
| B2Micro | 48 | 7 | 173 | 75.56 | (62.76, 88.77) |
| TFF3 | 0 | 6 | 177 | 60.55 | (37.76, 81.92) |
| TFF3 | 1 | 8 | 184 | 61.14 | (36.34, 83.9) |
| TFF3 | 2 | 8 | 185 | 48.38 | (23.78, 73.65) |
| TFF3 | 4 | 8 | 185 | 59.93 | (38.31, 80.88) |
| TFF3 | 8 | 8 | 180 | 50.42 | (23.47, 75.69) |
| TFF3 | 12 | 7 | 181 | 51.85 | (25.34, 77.59) |
| TFF3 | 24 | 7 | 180 | 66.67 | (41.51, 88.57) |
| TFF3 | 48 | 7 | 173 | 57.23 | (28.82, 82.99) |

The biomarkers A1Micro, B2Micro and TFF3 using the UCREA-normalization fold-change from baseline shows performance for distinguishing patients classified as “Risk” from patients classified as “No AKI”.

### Example 6

### Multivariate Assessment of Models

For the multivariate assessment of the models, we use different ways of combining the univariate markers into multivariate models, depending on the classification problem we use.

For the classification of “Injury” and “Failure” patients against “Risk” and “No AKI”, we use an approach that evaluates how different from a “normal” patient an observation is. In the first step, for every marker in the model, we estimate the distribution of the marker of the patients classified as “No AKI” using an approach that fits log-concave density functions. When evaluating a new observation, for every biomarker, the p-value with respect to the estimated distribution for “No AKI” patients is being evaluated. Then, the p-values are being combined by averaging them. Other options for combining the p-values we considered are taking the minimum, maximum or the average of the logarithm of the p-values. Each of these methods has certain trade-offs with respect to the sensitivity/specificity curve of the resulting models. Here smaller values of the risk score correspond to higher risk of having an AKI classified as “Injury” or “Failure”.

For classifying “Injury” or “Failure” against “Risk” or “No AKI”, the markers A1Micro, CLU, CYSC, IL-18, NGAL and TFF3 are being considered. We consider all possible combinations of these markers, but restrict to at most 3 markers at the same time. For each of these models, we calculate the classification performance at the time points 1 hour, 2 hours and 4 hours in terms of the AUC that the models achieve. Subsequently, we rank the models by averaging the 3 AUCs. In Table 1, find a list of all these models, sorted by the average of the AUC at time points 1 hour, 2 hours and 4 hours. The AUCs at these 3 time points are listed as well. The biomarker data used in this table has been transformed using the normalization with urinary creatinine.

TABLE 14
AUCs for time points 1 hour, 2 hours, 4 hours after arrival at
ICU for all combinations of at most 3 markers for classifying
“Injury” and “Failure” against “Risk” and “No AKI”.
Table is sorted by average of the AUCs at these 3 time points.

| | AUC- | AUC | AUC | AUC |
|---|---|---|---|---|
| Model | 1 h | -2 h | -4 h | -Ave. |
| TFF3 + A1Micro | 87.89 | 91.61 | 92.85 | 90.78 |
| TFF3 | 88.41 | 92.02 | 90.88 | 90.44 |
| A1Micro | 85.16 | 89.43 | 91.81 | 88.8 |
| CYSC + TFF3 + A1Micro | 86.07 | 89.33 | 90.47 | 88.62 |
| NGAL + TFF3 + A1Micro | 86.07 | 89.22 | 90.57 | 88.62 |
| TFF3 + CLU + A1Micro | 85.03 | 89.02 | 88.81 | 87.62 |
| IL-18 + TFF3 + A1Micro | 85.68 | 87.15 | 89.33 | 87.39 |
| CYSC + TFF3 | 85.55 | 88.5 | 87.67 | 87.24 |
| NGAL + TFF3 | 84.64 | 88.29 | 88.29 | 87.07 |
| CYSC + A1Micro | 83.2 | 86.53 | 87.36 | 85.7 |
| NGAL + A1Micro | 82.16 | 86.01 | 88.08 | 85.42 |
| IL-18 + TFF3 | 84.24 | 84.25 | 86.32 | 84.94 |
| TFF3 + CLU | 82.55 | 87.36 | 84.77 | 84.89 |
| CYSC + NGAL + TFF3 | 82.94 | 86.63 | 85.08 | 84.88 |
| NGAL + TFF3 + CLU | 82.03 | 85.91 | 84.56 | 84.17 |
| CYSC + NGAL + A1Micro | 81.38 | 85.08 | 85.39 | 83.95 |
| IL-18 + A1Micro | 81.51 | 82.59 | 87.46 | 83.85 |
| CYSC + TFF3 + CLU | 82.16 | 85.7 | 83.32 | 83.73 |
| IL-18 + NGAL + TFF3 | 82.29 | 83.63 | 85.18 | 83.7 |
| CLU + A1Micro | 80.21 | 85.39 | 85.18 | 83.59 |
| IL-18 + CYSC + TFF3 | 82.42 | 83.52 | 83.52 | 83.15 |
| IL-18 + NGAL + A1Micro | 80.86 | 82.69 | 85.08 | 82.88 |
| NGAL + CLU + A1Micro | 79.56 | 84.56 | 84.46 | 82.86 |
| CYSC + CLU + A1Micro | 79.95 | 84.77 | 83.63 | 82.78 |
| IL-18 + CYSC + A1Micro | 81.77 | 82.07 | 83.73 | 82.52 |
| NGAL | 78.91 | 82.49 | 83.21 | 81.54 |
| IL-18 + TFF3 + CLU | 79.95 | 82.07 | 82.49 | 81.5 |
| IL-18 + CLU + A1Micro | 78.39 | 81.45 | 83.32 | 81.05 |
| CYSC | 79.56 | 82.28 | 81.24 | 81.03 |
| CYSC + NGAL | 79.56 | 82.07 | 80.93 | 80.85 |
| CYSC + NGAL + CLU | 77.73 | 81.14 | 80.1 | 79.66 |
| NGAL + CLU | 77.08 | 80.31 | 80.62 | 79.34 |
| IL-18 + NGAL | 77.86 | 78.24 | 81.35 | 79.15 |
| CYSC + CLU | 77.99 | 34.80 | 78.76 | 78.92 |
| IL-18 + CYSC + NGAL | 77.6 | 79.17 | 79.9 | 78.89 |
| IL-18 + NGAL + CLU | 76.17 | 78.34 | 80.31 | 78.27 |
| IL-18 | 79.69 | 74.97 | 80.1 | 78.25 |
| IL-18 + CYSC | 77.47 | 77.51 | 79.69 | 78.22 |
| IL-18 + CYSC + CLU | 75.91 | 77.62 | 79.27 | 77.6 |
| IL-18 + CLU | 75.13 | 76.68 | 79.69 | 77.17 |
| CLU | 74.35 | 76.17 | 76.37 | 75.63 |

For the classification of patients in the “Risk” category against patients in the “No AKI” category, we consider two different models. In the first version, we take the biomarkers as they are after the transformation and average them. The resulting average of the markers is the risk score, with higher values corresponding to a higher risk of having an AKI. In the second version, each of the biomarkers is first standardized to have an average of 0 and a standard deviation of 1 on the group of “No AKI” patients. After this standardization, the markers in the models are averaged and this average is used as the risk score with again higher values corresponding to a higher risk of AKI. In Table 1, find a list of all these models, sorted by the average of the AUC at time points 1 hour, 2 hours and 4 hours. The AUCs at these 3 time points are listed as well. The biomarker data used in this table has been transformed using the normalization with urinary creatinine.

TABLE 15
AUCs for time points 1 hour, 2 hours, 4 hours after arrival at
ICU for all combinations of at most 3 markers for
classifying “Risk” against “No AKI”. Table is sorted by
average of the AUCs at these 3 time points.

| | AUC | AUC | AUC | AUC |
|---|---|---|---|---|
| Model | -1 h | -2 h | -4 h | -Ave. |
| TFF3 | 78.12 | 69.46 | 79.53 | 75.7 |
| TFF3 + A1Micro | 76.09 | 69.86 | 79.59 | 75.18 |
| A1Micro | 72.69 | 69.7 | 77.87 | 73.42 |
| TFF3 + A1Micro + B2Micro | 70.45 | 65.95 | 77.16 | 71.19 |
| TFF3 + B2Micro | 70.72 | 63.92 | 76.08 | 70.24 |
| A1Micro + B2Micro | 68.07 | 63.31 | 75.81 | 69.06 |
| B2Micro | 66.51 | 61.89 | 73.31 | 67.24 |

### Example 7

### Range of Assay by Time and Severity of AKI

Choice of markers was also made based on the dynamic range of the markers. In this example, the range of variability of the assay for IL-18, NGAL and TFF3, are shown for different AKI groups at different time points. For these plots, urinary creatinine normalized values were used.

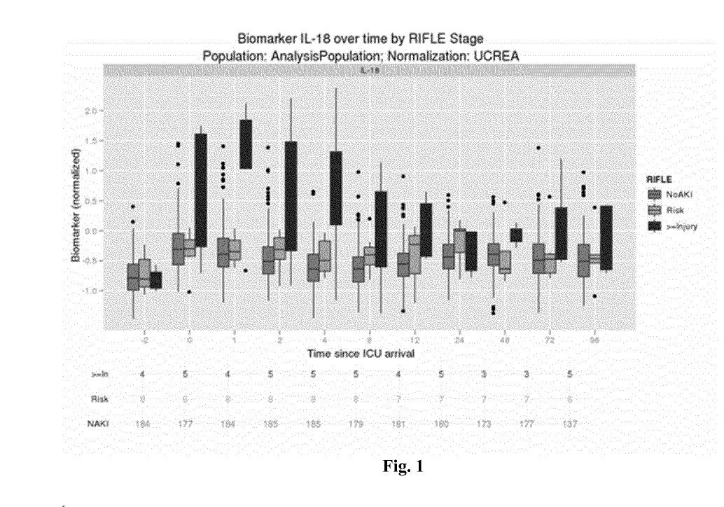

FIG. 1 shows a boxplot of IL-18 values after urinary creatinine normalization for different time points before and after surgery. The data shown is first transformed by taking the logarithm to base 10 before plotting. The plot illustrates fold changes in IL-18 of a factor of 100 and more when comparing patients with “Injury/Failure” to “No AKI” or “Risk” patients.

FIG. 1 shows a boxplot of NGAL values after urinary creatinine normalization for different time points before and after surgery. The data shown is first transformed by taking the logarithm to base 10 before plotting. The plot illustrates fold changes in NGAL of a factor of 10 and more when comparing patients with “Injury/Failure” to “No AKI” or “Risk” patients.

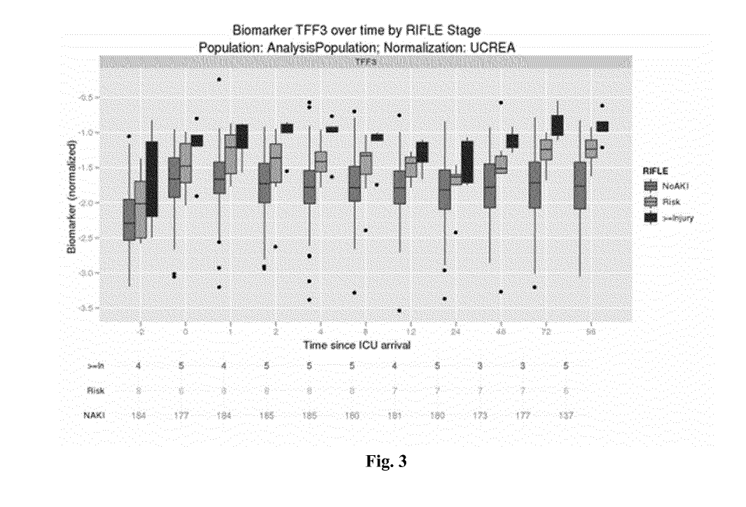

FIG. 3 shows a boxplot of TFF3 values after urinary creatinine normalization for different time points before and after surgery. The data shown is first transformed by taking the logarithm to base 10 before plotting. The plot illustrates fold changes in TFF of a factor of 3 and more when comparing patients with “Injury/Failure” to “No AKI” or “Risk” patients. Further the plot illustrates that TFF3 levels after surgery can discriminate “Risk” patients from “No AKI” patients better than other biomarkers, e.g. better than IL-18.

What is claimed is:

1. A method of assessing the severity of acute kidney injury (AKI) injury in a subject following cardiac surgery, comprising:
measuring one or more markers from Table 1 and/or Table 2 in a biological sample obtained from the subject within 24 hours following cardiac surgery;
generating a risk score based on the measured level of one or more of the biomarkers from Table 1, wherein if the risk score exceeds a predefined cutoff, the subject is determined to be at risk of developing RIFLE I/F; and
optionally, if the subject is not determined to be at risk of developing RIFLE I/F, further generating a risk score based on the measured level of one or more of the biomarkers selected from Table 2, wherein if the risk score exceeds a predefined cutoff, the subject is determined to be at risk of developing RIFLE R or if the risk score is below the predefined cutoff the subject is determined not to be at risk of developing AKI.

2. The method of claim 1, wherein two or more biomarkers from Table 1 are measured to determine if the subject is at risk of developing RIFLE I/F.

3. The method of claim 1, wherein three or more biomarkers from Table 1 are measured to determine if the subject is at risk of developing RIFLE I/F.

4. The method of claim 1, wherein two or more biomarkers from Table 2 are measured to determine if the subject is at risk of developing RIFLE R.

5. The method of claim 1, wherein the three biomarkers from Table 2 are measured to determine if the subject is at risk of developing RIFLE R.

6. The method of claim 1, wherein two or more biomarkers from Table 1 and Table 2 are measured to determine if the subject is at risk of developing RIFLE I/F or RIFLE R or no AKI.

7. The method of claim 1, wherein any of the biomarker combinations shown in Table 14 are measured to determine if the subject is at risk of developing RIFLE I/F.

8. The method of claim 1, wherein any of the biomarker combinations shown in Table 15 are measured to determine if the subject is at risk of developing RIFLE R.

9. (canceled)

10. A method of assessing the severity of acute kidney injury (AKI) injury in a subject following cardiac surgery, comprising:
measuring at least two of the following biomarkers selected from the group consisting of IL-18, Cystatin C, NGAL, TFF3, Clusterin, and A1-Microglobulin in a biological sample obtained from the subject within 24 hours following cardiac surgery; and
generating a risk score based on the measured level of the at least two biomarkers wherein the risk score is indicative if the subject is at risk of developing RIFLE I/F

11. (canceled)

12. A method of diagnosing or predicting development of acute kidney injury (AKI) in a subject following cardiac surgery, comprising measuring at least four of the following biomarkers selected from IL-18, Cystatin C, NGAL, TFF3, Clusterin, B2-microglobulin and A1-Microglobulin in a biological sample obtained from the subject within 24 hours following cardiac surgery; wherein the levels are indicative of AKI or are predictive of the development of AKI.

13.-14. (canceled)

15. The method of claim 1 further comprising measuring urinary creatinine (uCr) in the patient following CPB surgery and determining a ratio of each of the markers with uCr as a predictor of the development of acute kidney injury (AKI) in said patient.

16. The method of claim 1 further wherein the biomarker is measured between 0 hours and 12 hours following cardiac surgery.

17. The method of claim 1, wherein a weighted linear combination of at least one biomarker/uCr is used with Receiver-Operating Characteristic (ROC) area under the curve analysis is used to predict development of AKI in the subject.

18.-19. (canceled)
